(19) United States
(12) Patent Application Publication
(10) Pub. No.: US 2013/0165767 A1
(43) Pub. Date: Jun. 27, 2013

(54) SYSTEMS AND METHODS FOR AUTOMATIC LANDMARKING

(71) Applicants: Robert David Darrow, Scotia, NY (US); Ileana Hancu, Clifton Park, NY (US); Eric William Fiveland, Niskayuna, NY (US); William Thomas Dixon, Clifton Park, NY (US)

(72) Inventors: Robert David Darrow, Scotia, NY (US); Ileana Hancu, Clifton Park, NY (US); Eric William Fiveland, Niskayuna, NY (US); William Thomas Dixon, Clifton Park, NY (US)

(73) Assignee: GENERAL ELECTRIC COMPANY, SCHENECTADY, NY (US)

(21) Appl. No.: 13/332,977

(22) Filed: Dec. 21, 2011

(51) Int. Cl. A61B 5/055

(52) U.S. Cl. 600/414

(57) ABSTRACT

Embodiments of systems, methods, and non-transitory computer readable media for automatic landmarking are presented. A coil including at least one marker is placed on a desired region on interest (ROI) of a subject. Further, a detector detects a position of the marker while translating the subject from a home position in an imaging system into a bore of a magnet in the imaging system. A positioning unit coupled to at least one of the coil, the marker and the detector is configured to initiate automatic translation of the subject from the home position into the magnet bore. Further, the positioning unit determines a distance between the detected marker position and a homogenous position of the magnet based at least on the detected marker position. The positioning unit then controls movement of the subject over the determined distance to the homogenous magnet position for automatically landmarking the desired ROI.

### BACKGROUND

Diagnostic imaging procedures entail specific scan configurations that allow acquisition and reconstruction of imaging data from a desired region of interest (ROI) of a subject, such as a patient, to aid in accurate medical diagnoses. Magnetic Resonance (MR) imaging, for example, includes a plurality of scan configurations that specify parameters related to a patient position, positioning radio-frequency coils (RF) and landmarking an ROI of the patient for specific imaging protocols. In particular, landmarking registers a patient with a scanner coordinate system to allow an imaging volume to be moved to a homogeneous, imaging portion, for example, an iso-center of a magnet for desired imaging.

Typical landmarking is a manual process, in which a MR system operator defines the center of an imaging region through mechanical, optical, or other suitable means. By way of example, the system operator may position the patient on an examination table, position a MR coil on a desired ROI of the patient, followed by manually positioning the table within the magnet bore such that the desired ROI coincides with scanner alignment lights. The quality and consistency of landmarking and patient positioning, thus, mostly relies on the operator's skill and experience.

It may be noted that clinical decisions regarding diagnosis and treatment of disease conditions are often made based on certain image-derived parameters. Accordingly, accurate characterization of specific features of the anatomy of interest allows for a better understanding of patient anatomy and physiology, which in turn aids in early detection of various diseases. Consistency in manual landmarking, however, may be difficult for complex anatomies like the heart or joints, sometimes leading to incorrect patient positioning. Particularly, an error in judging the relative position of the coil by the operator may cause the center of the coil to be positioned at an offset from the iso-center to a less homogenous position of the magnet. Inaccurate estimations of clinically relevant parameters such as a location of a lesion derived from images reconstructed using erroneous configurations, thus, may lead to incorrect diagnosis, which in turn may adversely affect patient health.

Accordingly, accurate positioning of the anatomy of interest in the center of the magnet, characterized by the best field homogeneity, is significant in allowing for optimal imaging. A landmarking error, however, may result in images of low diagnostic quality and inadequate coverage of anatomy, thus making the images unsuitable, for example, for specific exams and/or automatic scan plane prescription algorithms. Particularly, inadequate coverage of the desired ROI may necessitate additional data acquisition in the direction of the superior/inferior (S/I) axis of the MR coil, thus further increasing the exam duration and adding to patient discomfort.

### BRIEF DESCRIPTION

Certain aspects of the present technique are drawn to systems, methods, and non-transitory computer readable media for automatic landmarking. The automatic landmarking entails placing a coil including at least one marker on a desired region on interest of a subject. The system further includes a detector configured to detect a position of the marker while translating the subject from a home position in an imaging system into a bore of a magnet in the imaging system. A positioning unit coupled to at least one of the coil, the marker and the detector is configured to initiate translation of the subject from the home position into the bore of the magnet automatically. Further, the positioning unit determines a distance between the detected position of the marker and a homogenous position of the magnet based at least on the detected position of the marker. The positioning unit then controls movement of the subject over the determined distance to the homogenous position of the magnet for automatically landmarking the desired region of interest.

### DRAWINGS

These and other features, and aspects of embodiments of the present technique will become better understood when the following detailed description is read with reference to the accompanying drawings in which like characters represent like parts throughout the drawings, wherein:

FIG. 1 is a pictorial view of an exemplary automatic landmarking system, in accordance with aspects of the present system; and

FIG. 2 is a flowchart depicting an exemplary method for automatic landmarking in an imaging system, in accordance with aspects of present technique.

### DETAILED DESCRIPTION

The following description presents exemplary systems and methods for automatic landmarking for non-invasive imaging of a subject. Particularly, embodiments illustrated hereinafter disclose automatic landmarking systems and methods that employ one or more coil markers and detectors to allow rapid and consistent patient landmarks in imaging systems.

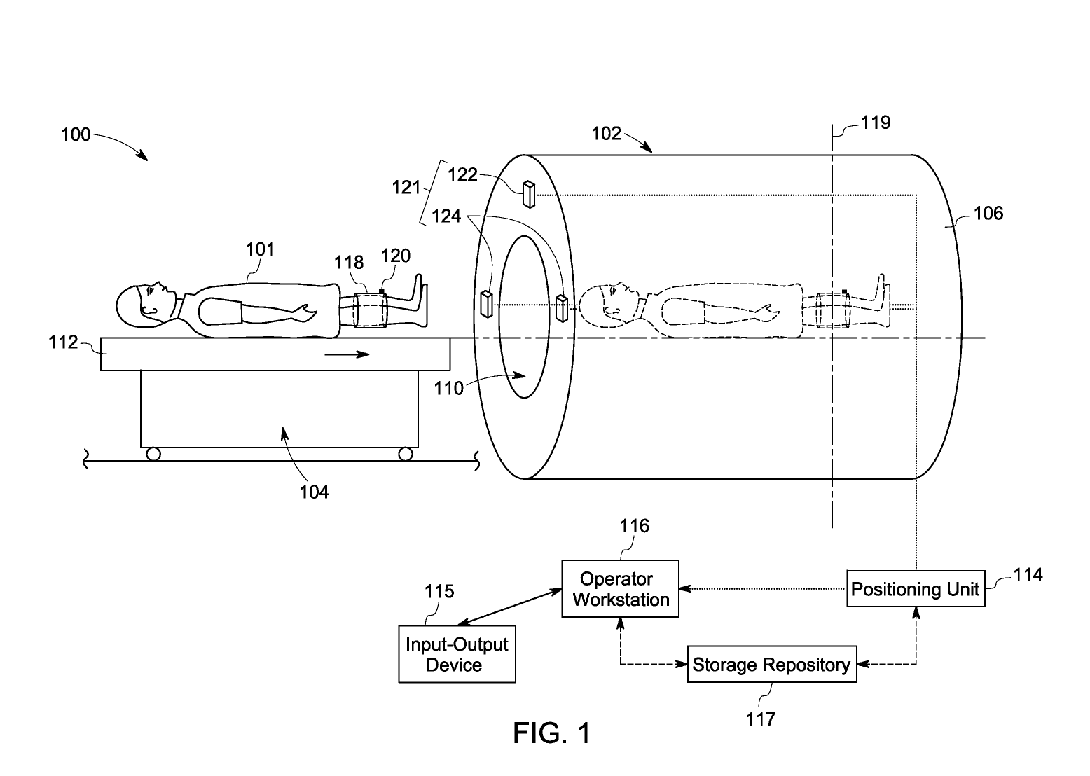

Although exemplary embodiments of the present technique are described in the context of a magnetic resonance imaging (MRI) operation, it will be appreciated that use of the present technique in various other imaging applications and systems is also contemplated. Some of these systems, for example, may include CT imaging systems, PET imaging systems, optical imaging systems, and hybrid systems combining MR with other modalities. An exemplary environment that is suitable for practicing various implementations of the present technique is discussed in the following sections with reference to FIG. 1.

FIG. 1 illustrates an exemplary system 100 for use in automatic landmarking of a subject, such as a patient 101. For discussion purposes, the system 100 is described with reference to patient preparation in an MR imaging operation. Accordingly, in one embodiment, the system 100 includes a magnetostatic field generator 102 operatively coupled to a motorized table unit 104. The magnetostatic field generator 102 includes a magnet 106, for example, including RF or gradient coils and a bore 110 to accommodate the patient 101, in one implementation, disposed in a supine position. In certain other implementations, however, the patient 101 may be disposed in other positions suitable for imaging.

To that end, the table unit 104 includes a cradle 112 that supports and translates the patient 101 into the magnet bore 110. Particularly, in certain embodiments, the table unit 104 includes a positioning unit 114 that governs motion of the cradle 112, and thus, the patient position within the magnet 106. The positioning unit 114, for example, governs the patient position based on operator inputs, specific exam requirements and/or designated scanning protocols. Accordingly, in certain embodiments, the positioning unit 114 includes devices such as one or more digital signal processors, microcomputers, microcontrollers, Application Specific Integrated Circuits (ASICs), Field Programmable Gate Arrays (FPGA), or one or more general-purpose or application-specific processors in communication with the system 100.

Particularly, in one embodiment, the positioning unit 114 controls the patient position based on operator inputs received through an input-output device 115 coupled to an operator workstation 116. The input-output device 115, for example, includes a display having a graphical user interface (GUI) or a switching subsystem for allowing the operator to select the desired scanning parameters and the desired ROI such as the patient's spine via the GUI. Alternatively, in certain embodiments, the positioning unit 114 controls the patient position based on scanning parameters specified in a configuration file received from a storage repository 117. To that end, the storage repository 117, for example, includes a random access memory, a read only memory, a disc drive, solid-state memory device, and/or a flash memory communicatively coupled to the system 100.

The positioning unit 114, on receiving the scanning parameters, automatically advances the cradle 112 into the bore 110, for example, using a table motor controller (not shown), and performs the landmark operation. To that end, the positioning unit 114 automatically detects a position of an RF coil 118 positioned on the desired ROI of the patient 101 under evaluation. In one embodiment, the RF coil 118 is positioned on a knee of the patient 101 before translating the patient 101 into the magnet bore 110. The patient 101 is then moved into the magnet bore 110 in a feet-first orientation. The patient 101, however, can be positioned in either the headfirst or the feet-first orientation depending upon the desired ROI of the patient 101 under evaluation. Brain exams, for example, may be performed with the patient 101 in the headfirst orientation, while lower chest and abdominal studies may be performed with the patient 101 in the feet-first orientation.

In a typical MR system, once the patient is positioned on the table, one or more laser alignment lights are turned on, and the operator moves the table into a magnet bore until the desired ROI is positioned under the laser alignment lights. The operator pushes a landmark button to register the patient with a coordinate system of the MR system and advances the patient to a scan position, which positions the region that was just landmarked in the center of the magnet, characterized by best field homogeneity. The operator then initiates patient scan by pushing a “start scan” button on an operator console. Typical MR imaging, thus, entails lengthy and multi-step landmarking operations that rely significantly on operator intervention, experience, and expertise.

However, unlike typical MR landmarking systems, the system 100 automatically landmarks, and scans the patient 101 based on a selected imaging protocol. In one embodiment, for example, the system 100 automatically landmarks and scans the knee of the patient 101 using a one-touch operation. To that end, the system 100 includes at least one marker 120 embedded or positioned on the RF coil 118 or placed directly on the region of interest of the patient 101 to allow for automatic landmarking of the desired ROI. The marker 120, for example, may be positioned at the S/I center, the S/I extents or any other suitable position on the RF coil 118. In another embodiment, multiple markers may be positioned along the length of the RF coil 118 allowing selective activation of one or more markers based on a scan prescription or configuration file. In a cardiac exam, for example, the positioning unit 114 selectively activates one or more markers disposed close to the heart of the patient 101.

Further, in certain embodiments, the marker 120 includes one or more passive and/or active elements. In one embodiment, for example, the marker 120 includes an infrared (IR) light emitting device (LED) disposed on the exterior of the RF coil 118. In another embodiment, the marker 120 includes a radio frequency identification (RFID) chip embedded in the RF coil 118. In a further embodiment, the marker 120 includes a secondary tuned RF coil attached to the RF coil 118. Additionally, in certain embodiments, where the marker 120 is implemented as an active device, the positioning unit 114 may control the marker 120 such that the marker 120 is activated during cradle travel to allow automatic landmarking, but is disabled during imaging.

Furthermore, in one embodiment, the system 100 also includes an emitter and/or detector assembly 121 for detecting the marker 120 while the patient 101 is moving into the magnet bore 110. To that end, the emitter-detector assembly 121, for example, may be mounted on a front surface or within the bore 110 of the magnet 106. Additionally, in certain implementations, the emitter-detector assembly 121 includes one or more active elements that elicit a response from the marker 120, in turn making the marker 120 acoustically and/or electromagnetically detectable.

In one embodiment, for example, a secondary tuned coil detects signal from the patient 101 or from a secondary source of signal, such as a vial filled with liquid attached to the tuned coil 120, only when it overlaps with the tuned RF coil marker 120. In this embodiment, either of the primary and the secondary tuned coils is of a design that increases precision of the localization via alignment of the coils. To that end, the primary or the secondary tuned coil may have a shape designed to generate a sharp null, for example a figure “8” shape, when the coils are aligned. In an alternative embodiment, however, the emitter-detector assembly 121 includes one or more passive elements to detect the marker 120, for example, an 8 millimeter reflective strip such as made from 3M Company's Scotchlite that includes high gain reflective sheeting.

To that end, in one example, the emitter-detector assembly 121 includes an emitter 122 such as an LED source directed towards the cradle 112 such that emitted radiation reflects off the reflective tape marker 120 and is detected by a detector 124 in the emitter-detector assembly 121. Particularly, in the present example, both the emitter 122 and the detector 124 may be mounted in parallel on the front surface of the magnet 106 such that resulting electromagnetic emissions reflect off the reflective tape marker 120 back to the detector 124 when the marker 120 reaches a detectable position. Additionally, in certain embodiments, the radiation may be modulated and encoded, for example, using a 38 kHz type modulation, and/or digital stream pickup of 1's and 0's to facilitate detection.

The positioning unit 114, on detecting the marker 120, configures one or more position encoders (not shown) associated with the table unit 104 to sense, for example, a longitudinal position of the cradle 112 within the magnet 106. In certain embodiments, a distance from the detector 124 to the iso-center 119 of the magnet 106 may be known. Alternatively, the positioning unit 114 may receive the distance information from a configuration file received from the storage repository 117 at the beginning or during the scan. The positioning unit 114 adds this distance to the measured cradle position to determine the distance of the cradle 112 to the iso-center 119 of the magnet 106.

In certain embodiments, the positioning unit 114 accounts for any offset of the marker 120 from the center of the RF coil 118 for computing the landmark position. In one implementation, for example, the marker 120 is positioned on one side of the RF coil 118 rather than in the center of the RF coil 118 for operational convenience. Accordingly, the positioning unit 114 determines the offset of the marker 120 from the center of the RF coil 118 and updates the value of the determined distance between the cradle 112 and the iso-center 119. The positioning unit 114 then advances the cradle 112 to the iso-center 119 and automatically sets a landmark to allow an advance to scan position of the desired ROI for generating diagnostic images for use in patient evaluation and treatment.

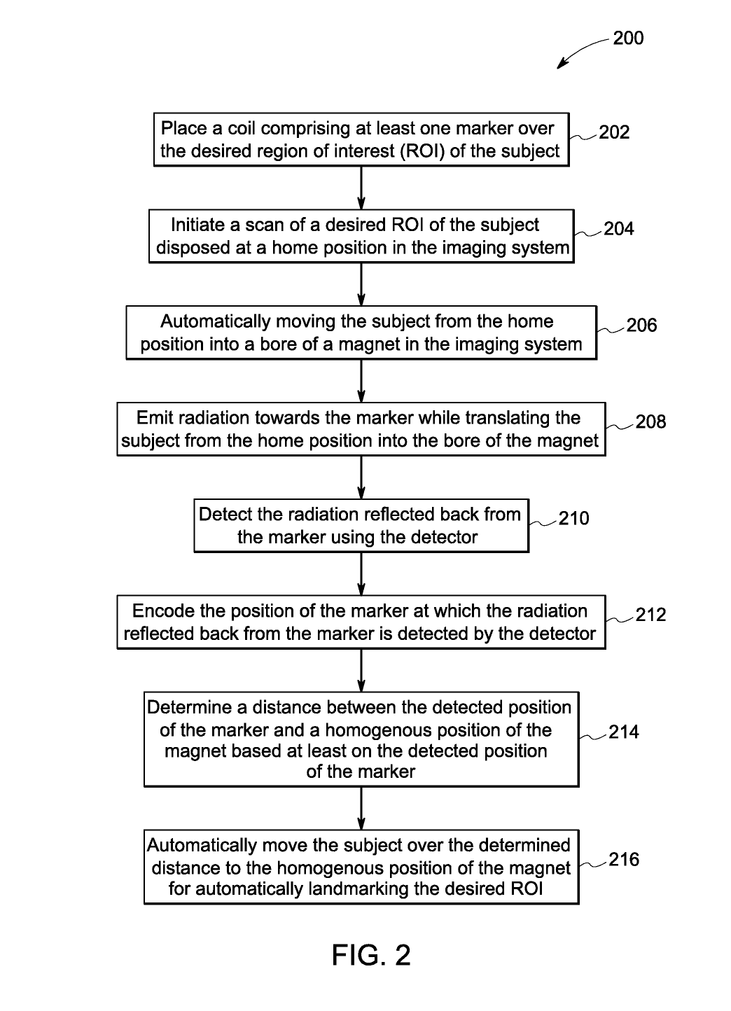

Embodiments of the landmarking system 100, thus, allow automatic landmarking of an anatomy of interest without any significant operator intervention. Particularly, the embodiments describe herein allow rapid, and consistent patient landmarking such that the center of an imaging coil is moved to the iso-center of the magnet precisely. Further, use of the embodiments of the present landmarking system may allow elimination of the laser alignment lights used in the manual landmark procedure, while also improving efficiency of an MR scanner by reducing the overall exam time. An exemplary method for automatic landmarking in MRI systems according to certain aspects of the present technique will be described in greater detail with reference to FIG. 2.

FIG. 2 illustrates a flow chart 200 depicting an exemplary method for automatic landmarking of a desired ROI in a MRI system using coil markers. The exemplary method may be described in a general context of computer executable instructions stored and/or executed on a computing system or at least one processor. Generally, computer executable instructions may include routines, programs, objects, components, data structures, procedures, modules, functions, and the like that perform particular functions or implement particular abstract data types.

Embodiments of the exemplary method may also be practiced in a distributed computing environment where optimization functions are performed by remote processing devices that are linked through a wired and/or wireless communication network, including cloud computing. In the distributed computing environment, the computer executable instructions may be located in both local and remote computer storage media, including memory storage devices.

Further, in FIG. 2, embodiments of the exemplary method are illustrated as a collection of blocks in a logical flow chart, which represents operations that may be implemented in hardware, software, or combinations thereof. The various operations are depicted in the blocks to illustrate the functions that are performed, for example, during initial calibration, coil marker detection, and automatic landmarking phases of the exemplary method. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the object matter described herein. For discussion purposes, the exemplary method will be described with reference to the elements of FIG. 1.

Accurate landmarking of an anatomy of interest in the center of a magnet, characterized by the best field homogeneity, is important to generate high quality images. Typical landmarking, however, entails a lengthy and manual pre-exam procedure, whose quality and consistency mostly relies on the operator's skill and experience. Accordingly, embodiments of the present method describe an exemplary technique for automatic landmarking of a desired ROI of a patient to generate one or more images for use in accurate diagnoses of patient health.

For discussion purposes, embodiments of the present method will be described with reference to automatic landmarking for MR imaging using the landmarking system 100 of FIG. 1. However, it may be noted, that embodiments of the present method can also be used in various other imaging applications and systems that require landmarking.

Accordingly, at 202, pre-exam preparation of a subject such as the patient 101 of FIG. 1 entails placing a coil including at least one marker over a desired ROI of a subject, such as the patient's knee. In one embodiment, the patient is positioned on the cradle of an imaging system such as an MRI system. Further, an appropriate RF coil is positioned to cover a ROI, that is, the knee of the patient under evaluation. However, in another embodiment, the patient is inserted into a body coil, for example, permanently mounted in the magnet bore, to selectively image different ROIs of the patient. In such a scenario, where the patient is positioned inside a fixed coil, one or more markers may be placed directly or indirectly on the patient.

To that end, the markers comprise one or more active and/or passive elements such as a reflective tape, an RFID, an IR LED and/or a tuned RF coil attached to the patient coil. In one embodiment, the detector includes a secondary tuned coil that detects a signal from the subject or a secondary source of signal only when coupling to the tuned radio frequency coil. Further, in one embodiment, the marker is either embedded in the coil, disposed on the exterior of the coil, and/or mounted at the S/I center of the patient coil. In an alternative embodiment, however, the marker is positioned at an offset from the center of the coil, for example, for operational convenience.

At step 204, the MR system or the operator initiates a scan of a desired ROI of the subject disposed at a home position in the imaging system, for example, by pressing a button available on the MR system or selecting a particular scan option via a GUI. As used herein, the term “home position” corresponds to a position of the cradle when it is completely outside the magnet bore. In one embodiment, the operator initiates the patient scan by selecting the desired ROI and specifying one or more scanning parameters, for example, a predetermined scanning prescription for imaging the desired ROI. In a further embodiment, initiating the patient scan loads a configuration file into the system memory. The configuration file, for example, includes predetermined or designated scanning parameters such as the distance between a detector and a homogenous position of a magnet in the imaging system.

Further, at step 206, the positioning unit begins translating the subject from the home position into a bore of a magnet in the imaging system automatically. Additionally, in certain embodiments, the positioning unit activates one or more emitters and detectors associated with the MR system for automatically landmarking the desired ROI of the patient. To that end, at step 208, an emitter disposed at a particular position of the MR system emits radiation towards the marker while the patient is being translated from the home position into the bore of the magnet.

Further, at step 210, one or more detectors disposed on the MR system detect the radiation reflected back from the marker during patient translation. In certain embodiments, the IR detection is calibrated during an initial imaging operation by an automatic landmarking of the coil, followed by manually returning to the landmark position, where the cradle position is adjusted until the laser alignment lights coincide with the center of the coil. The offset, thus determined, is used as the landmark error and may be accounted for while determining the distance by which the cradle needs to move to be positioned at the iso-center of the magnet.

Accordingly, at step 212, the positioning unit configures one or more encoders associated with the cradle to encode the position of the marker at which the radiation reflected back from the marker is detected by the detector. To that end, in one embodiment, the positioning unit receives the radiation information detected by the detectors and further filters the received information to generate digital signals for transmission to the encoders. The encoders evaluate the digital signals to determine the position of the cradle at the instant of time at which the radiation reflected back from the marker is detected by the detector.

The detected position of the marker, at step 214, is then used to determine a distance between the detected position of the marker indicative of the cradle position within the magnet, and the center of the magnet. In one embodiment, the positioning unit determines the distance between the detected position of the marker and the homogenous position of the magnet by adding the detected position of the marker to a predetermined distance between the detector and the homogenous position of the magnet. The predetermined distance, for example, may be stored in the storage repository or in the configuration file loaded at the beginning of the MRI exam.

Positioning the desired ROI at the homogenous position of the magnet aids in accurate and consistent imaging of the desired ROI. Accordingly, in certain embodiments, where the marker is disposed at an offset from the center of the patient coil, the positioning unit updates the determined distance to account for the offset. Further, at step 216, the positioning unit configures the cradle to move the subject over the determined distance to the homogenous position of the magnet for automatically landmarking the desired ROI.

Embodiments of the present methods and systems, thus, disclose an efficient landmarking technique that eliminates tedious and inconsistent manual landmarking procedures for MRI systems. Particularly, embodiments of the present methods and systems described herein allow rapid and accurate landmarking of one or more desired ROIs of a patient without using complicated image processing or manual intervention. Furthermore, certain embodiments of the present system provide sub-millimeter precision by accurately landmarking the ROIs to position the ROI at the iso-center of the magnet for optimal imaging even when using uncomplicated and inexpensive components such as reflective tapes IR emitters and detectors. Additionally use of the embodiments of the present landmarking system may allow elimination of certain elements such as the laser alignment lights used in the manual landmark procedure, while also improving efficiency of an MR scanner by reducing an overall exam time.

Although specific features of various embodiments of the invention may be shown in and/or described with respect to some drawings and not in others, this is for convenience only. It is to be understood that the described features, structures, and/or characteristics may be combined and/or used interchangeably in any suitable manner in the various embodiments, for example, to construct additional assemblies and techniques. Further, while only certain features of the present invention have been illustrated and described herein, many modifications and changes will occur to those skilled in the art. It is, therefore, to be understood that the appended claims are intended to cover all such modifications and changes as fall within the true spirit of the invention.

1. A system, comprising:
a coil placed on a desired region on interest of a subject, wherein the coil comprises at least one marker;
a detector configured to detect a position of the marker while translating the subject from a home position in an imaging system into a bore of a magnet in the imaging system; and
a positioning unit coupled to at least one of the coil, the marker and the detector, wherein the positioning unit is configured to:
initiate translation of the subject from the home position into the bore of the magnet automatically;
determine a distance between the detected position of the marker and a homogenous position of the magnet based at least on the detected position of the marker; and
control movement of the subject over the determined distance to the homogenous position of the magnet for automatically landmarking the desired region of interest.

2. The system of claim 1, further comprising an emitter configured to emit radiation towards the detector while translating the subject from the home position into the bore of the magnet.

3. The system of claim 2, wherein the emitter is configured to modulate and encode the radiation to improve detectability.

4. The system of claim 3, wherein the emitter is configured to modulate and encode the radiation using a 38 kHz type modulation, digital stream pickup of 1's and 0's, or a combination thereof, to improve detectability.

5. The system of claim 2, wherein the detector is configured to detect the radiation reflected back from the marker.

6. The system of claim 5, further comprising one or more encoders configured to encode the position of the marker at which the radiation reflected back from the marker is detected by the detector.

7. The system of claim 1, wherein the marker comprises one or more active marker elements.

8. The system of claim 1, wherein the marker comprises one or more passive marker elements.

9. The system of claim 1, wherein the marker comprises a reflective tape.

10. The system of claim 1, wherein the marker comprises a radio frequency identification device.

11. The system of claim 1, wherein the marker comprises an infrared light emitting device disposed on an exterior surface of the coil.

12. The system of claim 1, wherein the marker comprises a tuned radio frequency coil attached to the coil.

13. The system of claim 12, wherein the detector comprises a secondary tuned coil that detects a signal from the subject or a secondary source of signal only when coupling to the tuned radio frequency coil.

14. The system of claim 12, wherein either of the coil and the tuned radio frequency coil has a shape configured to increase precision of localization when the coil and the tuned radio frequency coil are aligned.

15. The system of claim 12, wherein either of the coil and the tuned radio frequency coil has a shape configured to generate a sharp null when the coils are aligned.

16. The system of claim 15, wherein either of the coil and the tuned radio frequency coil has a figure “8” shape.

17. The system of claim 1, wherein the marker is positioned at the superior inferior center of the coil.

18. The system of claim 1, wherein the coil comprises a plurality of markers.

19. The system of claim 18, wherein the positioning unit is configured to selectively activate one or more of the plurality of markers based on a scan prescription or configuration file. The system of claim 1, wherein the positioning unit is configured to activate the marker while translating the subject from a home position in an imaging system into the bore of the magnet.

20. The system of claim 1, wherein the positioning unit is configured to deactivate the marker while imaging the desired region of interest of the subject.

21. The system of claim 1, further comprising an input device for selecting the desired region of interest, specifying one or more scanning parameters for imaging the desired region of interest, or a combination thereof.

22. The system of claim 1, wherein the system is a magnetic resonance imaging system.

23. A method for automatic landmarking in an imaging system, comprising:
initiating a scan of a desired region of interest of a subject disposed at a home position in the imaging system, wherein a coil comprising at least one marker is placed over the desired region of interest of the subject;
automatically moving the subject from the home position into a bore of a magnet in the imaging system;
detecting a position of the marker while translating the subject from the home position into the bore of the magnet using a detector;
determining a distance between the detected position of the marker and a homogenous position of the magnet based at least on the detected position of the marker; and
automatically moving the subject over the determined distance to the homogenous position of the magnet for automatically landmarking the desired region of interest.

24. The method of claim 23, wherein automatically initiating the scan of the desired region of interest of the subject comprises selecting the desired region of interest.

25. The method of claim 23, wherein automatically initiating the scan of the desired region of interest of the subject comprises specifying one or more scanning parameters for imaging the desired region of interest.

26. The method of claim 23, wherein detecting the position of the marker comprises:
emitting radiation towards the marker while automatically moving the subject from the home position into the bore of the magnet.
detecting the radiation reflected back from the marker using the detector; and
encoding the position of the marker at which the radiation reflected back from the marker is detected by the detector.

27. The method of claim 19, wherein determining the distance between the detected position of the marker and the homogenous position of the magnet comprises adding the detected position of the marker to a predetermined distance between the detector and the homogenous position of the magnet.

28. The method of claim 27, wherein determining the distance between the detected position of the marker and the homogenous position of the magnet comprises updating the determined distance based on an offset between a position of the marker on the coil and the center of the coil.

29. The method of claim 27, further comprising:
calibrating a landmark operation by manually moving the subject towards the homogenous position of the magnet until one or more laser alignment lights in the imaging system coincide with the center of the coil;
determining a landmark error based on a distance by which the subject is manually moved towards the homogenous position of the magnet until one or more laser alignment lights in the imaging system coincide with the center of the coil; and
updating the determined distance based on the determined landmark error.

30. A non-transitory computer readable medium that stores instructions executable by one or more processors to perform a method for automatic landmarking in an imaging system, comprising:
initiating a scan of a desired region of interest of a subject disposed at a home position in the imaging system, wherein a coil comprising at least one marker is mounted over the desired region of interest of the subject;
automatically moving the subject from the home position into a bore of a magnet in the imaging system;
detecting a position of the marker while translating the subject from the home position into the bore of the magnet using a detector;
determining a distance between the detected position of the marker and a homogenous position of the magnet based at least on the detected position of the marker; and
automatically moving the subject over the determined distance to the homogenous position of the magnet for automatically landmarking the desired region of interest.
